(19) United States
(12) Patent Application Publication
(10) Pub. No.: US 2018/0014755 A1
(43) Pub. Date: Jan. 18, 2018

(54) PORTABLE DEVICE, METHOD AND PROGRAM PRODUCT FOR GENERATING A PARAMETER RELATED TO THE PHYSICAL ACTIVITY OF A USER

(71) Applicant: TECHNOGYM S.p.A., Cesena, Forli'-Cesena (IT)

(72) Inventors: Nerio ALESSANDRI, Cesena, Forli'-Cesena (IT); Silvano ZANUSO, Cesena, Forli'-Cesena (IT)

(73) Assignee: TECHNOGYM S.p.A., Cesena, Forli'-Cesena (IT)

(21) Appl. No.: 15/547,010

(22) PCT Filed: Jan. 26, 2016
(86) PCT No.: PCT/IB2016/050374

§ 371 Date: Jul. 27, 2017

Related U.S. Application Data
(63) Continuation-in-part of application No. 14/610,450, filed on Jan. 30, 2015.

(30) Foreign Application Priority Data
Apr. 30, 2015 (IT) MI2015A000627

(51) Int. Cl. A61B 5/11; A61B 5/0402; A61B 5/044; G06F 19/00; A61B 5/00

(52) U.S. Cl. CPC A61B 5/1118; G06F 19/3481; G06F 19/3406; A61B 5/6898; A61B 5/044; A61B 5/04028; A61B 5/4866; A61B 2562/0219

(57) ABSTRACT

A portable device (1), method and program product eneratea parameter related to the physical activity of a user. The device (1) includes a sensor (2) configured to measure information representative of the movement of the user. The sensor (2) is configured to generate, on the basis of such information, values of physical activity data. A data processing unit (3) operatively connected to the sensor (2), is configured to generate at least one physical activity rate parameter on the basis of the values of physical activity data generated by the sensor (2). The data processing unit (3) generates the at least one physical activity rate parameter in a first reference time instant on the basis of a portion of values of the values of physical activity data generated by the sensor (2) in a set period of time preceding the first reference time instant.

### FIELD OF THE INVENTION

The present invention relates to the health and the physical activity of a user, particularly to a portable device, method and program product for generating a parameter related to the physical activity of a user.

### TECHNOLOGICAL BACKGROUND

Nowadays, many people are aware of the importance of healthy nutrition and sufficient physical activity.

In this regard, portable devices are known which are able to monitor the physical activity of a user, generating information representative of the performed physical activity.

Examples of such portable devices comprise a sensor (e.g. an accelerometer) configured to collect information representative of the movement of the user and a processing unit configured to generate and provide the user with information representative of the amount of the physical activity performed by the user.

However, nowadays, such portable devices and the information they are able to provide the user with, are no longer sufficient or satisfactory.

Therefore, making available portable devices able to provide the user with more information about the performed physical activity in order to stimulate the physical activity of a user in a more precise and reliable manner is strongly needed.

### SUMMARY OF THE INVENTION

It is the object of the present invention to provide a portable device which allows to overcome the drawbacks of the prior art mentioned above, in particular which can provide the user with more precise and reliable parameters related to the physical activity performed by the user.

Such an object is achieved by a portable device according to claim 1.

The present invention further relates to a method for generating a parameter related to the physical activity of a user.

The present invention also relates to a program product loadable in a memory unit of an electronic calculator (e.g. an electronic device, such as a smartphone, a tablet, a PDA, a smartwatch and so on), the program product being executed by a data processing unit of the electronic calculator.

### BRIEF DESCRIPTION OF THE DRAWINGS

Further features and advantages of the portable device, the method and program product according to the present invention will become more apparent from the following description of preferred embodiments thereof, given by way of a non-limiting example, with reference to the accompanying drawings, in which:

FIG. 1 shows a diagrammatic view of a portable device according to an embodiment of the invention;

FIG. 2 shows a block diagram representative of a method for generating a parameter related to the physical activity of a user according to an embodiment of the invention;

FIGS. 3, 4, 5, 6, 9 and 10 show graphic elements which can be viewed on the display of the portable device in FIG. 1;

FIGS. 7 and 8 diagrammatically show a portable device adapted to show on the display graphic elements according to two different operating conditions of the portable device.

### DETAILED DESCRIPTION OF THE PREFERRED EMBODIMENTS

Similar or equivalent elements in the figures are indicated with the same reference numerals and/or alphanumeric references.

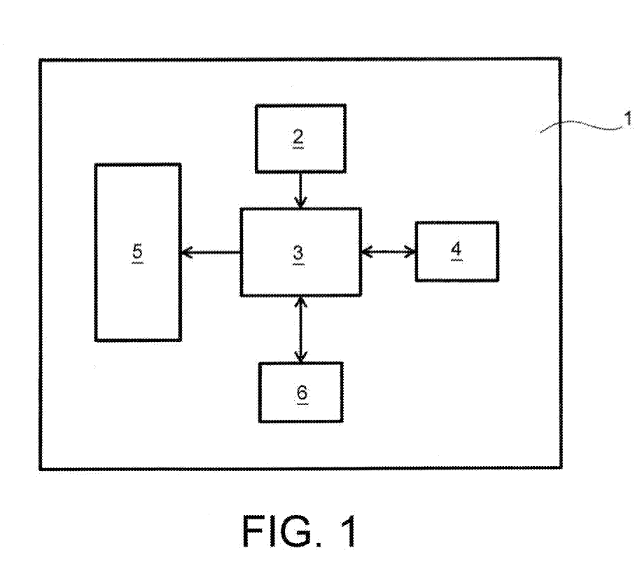

With reference to the embodiment of FIG. 1, a portable device 1 for generating a parameter related to the physical activity of a user, hereinafter also referred to simply as “device”, is now described. Such a parameter will be described below.

It should be observed that in the embodiment of FIG. 1, the device 1 is represented as a block diagram only, in order to better highlight the technical features of the device that are important for the present disclosure.

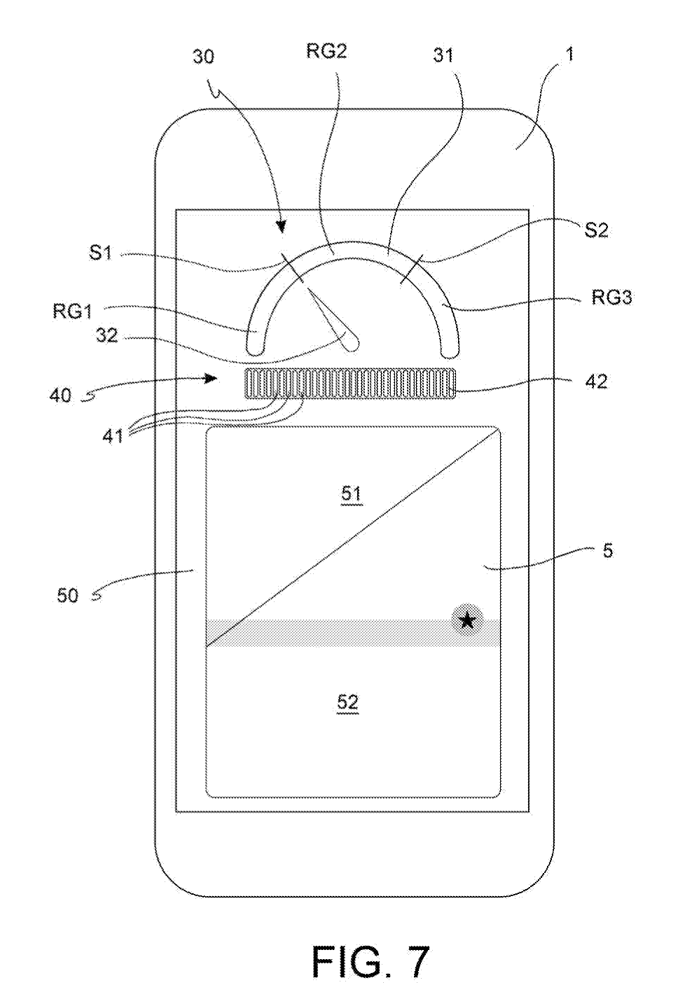

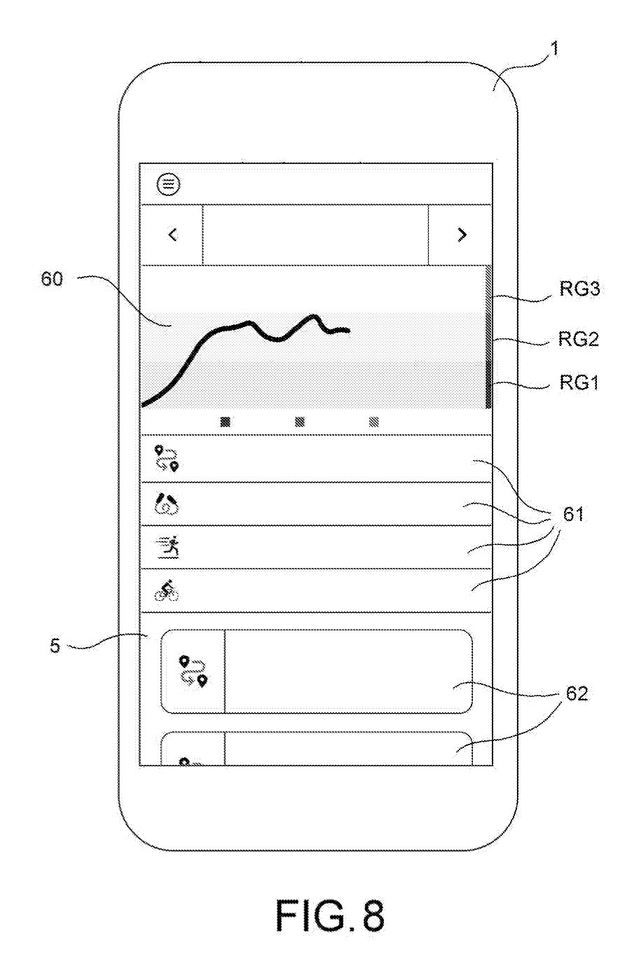

The device of FIG. 1 can be a smartphone (as shown inter alia in FIG. 7 and FIG. 8), a tablet, a smartwatch, a personal digital assistant (PDA), a multimedia digital player or any other electronic apparatus which can be worn by, or which may equip a user.

The device 1 comprises at least one sensor 2 to measure information representative of the movement of the user.

For the purposes of the present invention, “information representative of the movement of the user” means information related to the movement of the user's body during the physical activity performed while training or during normal everyday life, such as for example acceleration, speed and so on.

Such as least one sensor 2 is configured to generate, on the basis of such information, a plurality of values of physical activity data.

In greater detail, the physical activity data generated on the basis of such information relate to the intensity of the physical activity performed by the user.

With this regard, it should be noted that such intensity of the physical activity may be expressed by the physiological measure expressing the energy cost of physical activities called MET (i.e. METabolic equivalent).

Turning back to FIG. 1, the at least one sensor 2 can be an accelerometer (2D or 3D version), a gyroscope, a combination of the two, or any other electronic component able to measure information representative of the movement of the user and to generate, on the basis of such information, a plurality of values of physical activity data (MET).

Turning back to FIG. 1, the device 1 further comprises a data processing unit 3, e.g. a microprocessor or a microcontroller, operatively connected to said at least one sensor 2, configured to generate at least one activity index parameter on the basis of a plurality of physical activity data values generated by said at least one sensor 2.

In addition, the data processing unit 3 is further configured to generate a physical activity rate parameter in a first reference time instant t0 on the basis of a portion of values of the plurality of values of physical activity data generated by said at least one sensor 2 in a set period of time preceding the first reference time instant to.

As will be explained below, “first reference time instant t0” means the time instant in which the data processing unit 3 generates the physical activity rate parameter.

The set period of time preceding the first reference time instant t0 may be set as any period of time preceding the first reference time instant t0, e.g. 1 hour before the first reference time instant t0, half hour before the first reference time instant t0, 10 minutes before the first reference time instant t0, one minute before the first reference time instant t0, and so on.

According to a further embodiment, the set period of time preceding the first reference time instant t0 is a period of time comprised between a time instant of beginning of physical activity and the first reference time instant t0.

“Time instant of beginning of physical activity” means the time instant in which said at least one sensor 2 generates a set number of values of physical activity data in a set period of beginning of physical activity. By way of example, the set number values of physical activity data may be 5, 6, 7 and so on, generated in a set period of beginning of physical activity equal to three minutes.

It is further worth noting that the time instant of beginning of physical activity may be stated by the user according to his or her habits. For example, a user who wakes up every morning at 7 o'clock can set that time as time instant of beginning of physical activity.

On a general level, the fact of generating a physical activity rate parameter in a first reference time instant t0 on the basis of a portion of values of physical activity data generated by said at least one sensor 2 in a set period of time preceding the first reference time instant t0 advantageously allows to obtain a physical activity rate parameter representative of the physical activity of the user in a set period of time preceding the time instant in which such a parameter is generated (the first reference time instant t0), such as the last hour, the last half hour, the last ten minutes, the entire period during which the physical activity rate was detected by the user.

In such a manner, it is thus possible to provide the user, in any time instant, with an updated parameter related to the physical activity rate performed by the user herself/himself, who can thus obtain relevant information in more accurate and reliable manner.

Turning again to the embodiment of FIG. 1, the device 1 further comprises a memory unit 4, operatively associated to the data processing unit 3. The memory unit 4 is configured to store both a program product (e.g. program code or a client software application) to allow the device 1 to generate the parameter related to the physical activity of the user, as described below, and the data processed by the data processing unit 3 during the operation of the device 1.

In particular, the memory unit 4 is configured to store the plurality of values of physical activity data generated by said at least one sensor 2 and obviously also the physical activity rate parameter generated by the data processing unit 3.

In addition, the memory unit 4 is configured to store one or more client software applications to allow the user to enjoy one or more digital services provided by one or more client software applications directly through the device 1.

Furthermore, it is also confirmed that “client software application” means client software application of the standard type available on the market for electronic calculators and/or portable electronic devices, such as a personal computer, a notebook, a mobile phone (e.g. a smartphone), a tablet, a digital audio/video file reader (e.g. a MP3 reader), and so on.

The device 1 further comprises a display 5, operatively connected to the data processing unit 3, configured to display to the user at least one of the following: the plurality of values of physical activity data generated by the sensor 2 and the activity index parameter generated by the data processing unit 3.

The device 1 may also comprise a control panel, i.e. a keyboard or a graphical user interface, (not shown in FIG. 1) configured to allow the user to interact with the portable device 1. In the case the control panel was of the touch screen type, the control panel corresponds to the display 5.

The device 1 may also comprise a data communication module 6 configured to communicate with other electronic devices and electronic calculators (e.g. a server computer), also arranged in a remote location, via a data communication channel in a wired or wireless mode.

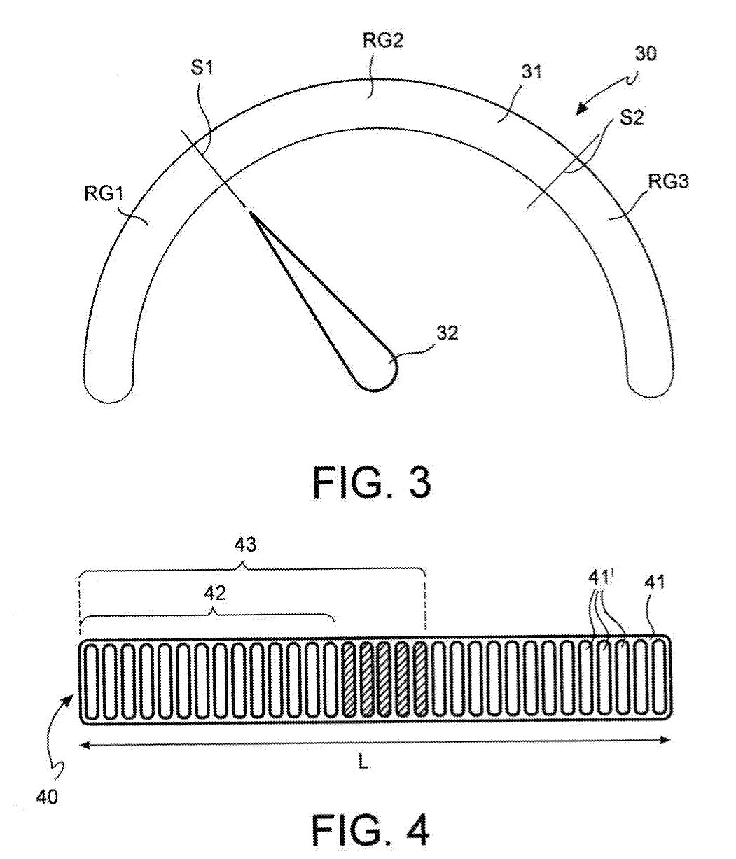

With reference now also to FIGS. 3, 4, 5 and 6, it is worth noting that the data processing unit 3 is configured to generate one or more graphic elements to be displayed on the display 5.

A graphic element may be representative of the plurality of values of physical activity data generated by said at least one sensor 2, of the physical activity rate parameter generated by the data processing unit 3, of a combination thereof, or of other information related in all cases to the physical activity performed by the user.

With reference in particular to FIG. 3, the data processing unit 3 is configured to generate a first graphic element 30 representative of the generated physical activity rate parameter. In the example of FIG. 3, the first graphic element 30 comprises a dashboard 31 and an indicator 32.

In greater detail, the data processing unit 3 is configured to compare the generated physical activity rate parameter with a set plurality of threshold values of the generated physical activity rate parameter. The set plurality of threshold values is adapted to define a plurality of value intervals of said physical activity rate parameter, each of such intervals being representative of a qualitative index of the represented physical activity rate parameter.

With particular reference to FIG. 3, such a plurality of threshold values may comprise at least one first threshold value and one second threshold value, indicated by S1 and S2 in the figure, so as to define at least three intervals into which dividing the dashboard 31, a first interval RG1, having the first threshold value S1 as upper value, a second interval RG2, comprised between the first threshold value S1 and the second threshold value S2, a third interval RG3, having the second threshold value S2 as lower extreme.

The data processing unit 3 is configured to position the indicator 32, relatively to the dashboard 31, on the basis of the comparison of the generated physical activity rate parameter with the set plurality of threshold values of the generated physical activity rate parameter, e.g. on hourly basis, as will be explained below.

In the embodiment of FIG. 3, if the generated physical activity rate parameter is under the first threshold value S1, the indicator 32 will be positioned at the respective value within the first interval RG1. From the quality point of view, the indicator 32 indicates to the user that the intensity of the physical activity is “low”, comparable to that of a sedentary user.

If the generated physical activity rate parameter is comprised between the first threshold value S1 and the second threshold value S2, the indicator 32 will be positioned at the respective value within the second interval RG2. From the quality point of view, the indicator 32 indicates to the user that the intensity of the physical activity is “medium” or “active”, comparable to that of an active, but not sporty, user.

If the generated physical activity rate parameter is above the second threshold value S2, the indicator 32 will be positioned at the respective value within the third interval RG3. From the quality point of view, the indicator 32 indicates to the user that the intensity of the physical activity is “high” or “sporty”, i.e. comparable to that of a sporty user.

From a graphic point of view, in order to qualitatively facilitate the reading of the first graphic element 30 by the user, the dashboard 31 may be, for example, a red-yellow-green color scale passing from the first interval RG1 to the third interval RG3 and/or displaying alphanumeric writing (not shown in FIG. 3) at each defined value interval.

With reference now to FIGS. 4, 9 and 10, the data processing unit 3 is further configured to generate a second graphic element 40 representative of the amount of values of physical activity data generated by said at least one sensor 2.

With this regard, the second graphic element 40 comprises a reference bar 41, whose length L is representative of the target determined by the user in a set period of time (e.g. daily target). It is worth noting that such target is previously generated by the data processing unit 3 on the basis of user physical activity data generated by said at least one sensor 2 in a previously set period of time, e.g. during the previous two weeks.

The data processing unit 3 is configured to display on the reference bar 41 a first progressive bar 42 representative of the amount of physical activity data values generated by said at least one sensor 2 in a set current time instant tc (t0). This occurs, for example, by illuminating and/or coloring with a first color (e.g. white) a portion of the reference bar 41 corresponding to the first progressive bar 42. In an embodiment, shown in FIG. 4, the reference bar 41 comprises a plurality of bars 41′ adapted to be illuminated and/or colored in order to represent the first progressive bar 42.

Furthermore, the data processing unit 3 is configured to display on the reference bar 41 a second progressive bar 43 representative of the amount of values of physical activity data generated by said at least one sensor 2 at the same current time instant tc (t0) of a preceding set period of time (e.g. the last two weeks). This occurs, for example, by illuminating and/or coloring with a second color (e.g. grey) a portion of the reference bar 41 corresponding to the second progressive bar 42. In an embodiment, shown in FIG. 4, the reference bar 41 comprises a plurality of bars 41′ adapted to be illuminated and/or colored in order to represent the second progressive bar 43.

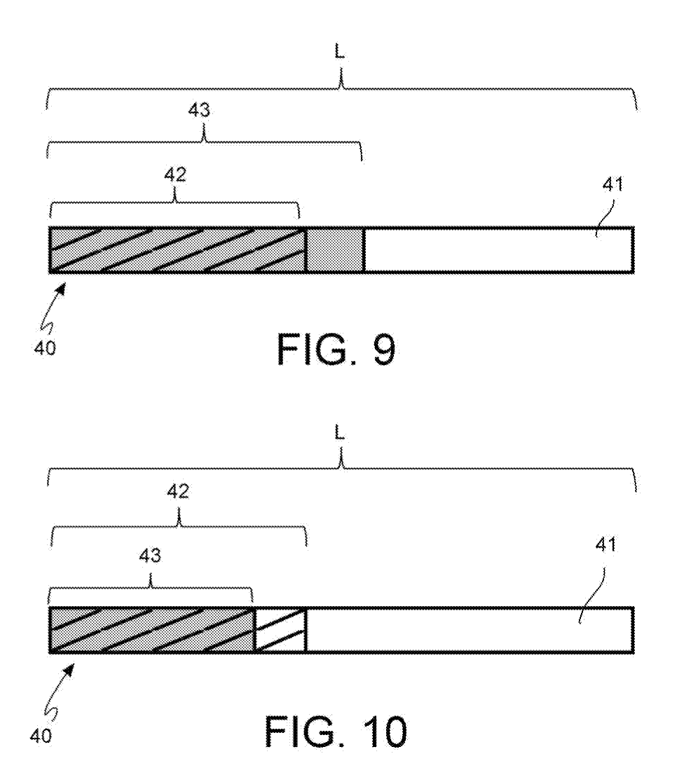

In the example of FIG. 9, the second progressive bar 43 is in excess with respect to the first progressive bar 42.

In the example of FIG. 10, the second progressive bar 43 is in defect with respect to the first progressive bar 42.

In other words, the second number of bars of said plurality 41 represents an average amount of physical activity data generated by the user in the same current time instant, but generated in a previous set period of time (e.g. in the last two weeks). For example, if the current time instant is a specific time (10 o'clock), the second number of bars of said plurality 41 represents an average amount of physical activity data generated in the same specific time period of the past two weeks.

This allows the user to view on the portable device 1 the generated physical activity data in the current instant not only with respect to the set target but also with respect to the physical activity data generated in a previous set period of time. This allows the user to determine whether he or she is improving the previous performance in the current time instant, regardless of whether the set target is being reached or not.

Furthermore the main advantage for the user is that the value of the second progressive bar 43 represents a “reference” to be followed to attempt to achieve and reach the target of “amount of values of physical activity data” at the end of the day (which is represented by the total length of the reference bar 41). In this manner, the user can predict whether the target will be reached or not and in all cases whether it is coherent with what was previously performed in view of reaching the target. If the user is further ahead (first progressive bar 42 greater than the second progressive bar 43) the user knows that he or she is improving the previous performance with respect to the average of the previous period.

It should be noted that in an embodiment, not shown in the figures, the data processing unit 3 is configured to show a first numeric data representative of the physical activity data generated by said at least one sensor 2 and a second numeric data representative of the target determined by the user, close to the second graphic element 40.

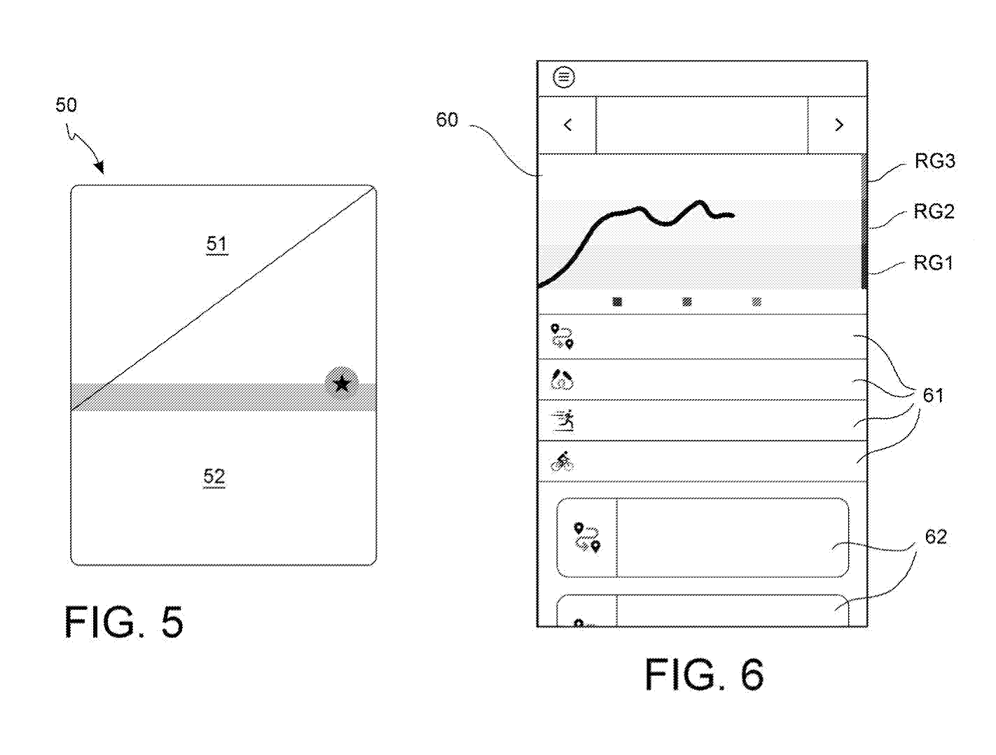

With reference now to FIG. 5, the data processing unit 3 is further configured to generate a third graphic element 50 representative of an information sheet adapted to suggest to the user a physical exercise or how to improve lifestyle (e.g. nutritional advice or a suggestion to perform an outdoor activity or an indoor activity, on the basis of where the user is located by the device 1 and also on the basis of other conditions (e.g.: the weather)). For example, if the user is at office, the sheet can suggest physical stretching exercises; if the user is at home and it is sunny, the sheet may suggest an outdoor run, and if it is raining the sheet may suggest indoor physical exercises.

In greater detail, such a sheet comprises a first portion 51, in which an image representative of the physical exercise to be performed can be inserted, and a second portion 52, in which an alphanumerical descriptive text of the exercise and of the physical activity data to be obtained by the user can be inserted.

With reference now to FIG. 6, the data processing unit 3 is further configured to generate a fourth graphic element 60 representative of the amount of physical activity data generated by said at least one sensor 2 during a set overall period of time, e.g. during one day.

In greater detail, the fourth graphic element is a time diagram which shows the time on the abscissa axis and the physical activity rate parameter on the ordinate axis. The amount of physical activity data generated over time can be placed within one of the physical activity rate intervals RG1, RG2, RG3 shown diagrammatically also in FIG. 6, described above with reference to FIG. 3.

Again with reference to FIG. 6, it is worth noting that the data processing unit 3 is configured to generate a first portion of further graphic elements 61, close to the fourth graphic element 60, each of which is representative of a physical exercise or physical activity which can be performed by the user, together with the target set by the user with regards to the physical activity data to be generated and to a set period of time of performance of the physical exercise or physical activity.

Furthermore, the data processing unit 3 is configured to generate a second portion of further graphic elements 62, each of which is representative of a physical exercise of physical activity which can be performed by the user, together with the progressive value of generated physical activity data and with the incremental value of the time of performance of the physical exercise or physical activity.

With reference to FIGS. 7 and 8, the graphic elements which can be generated by the data processing unit 3 of the portable device 1 are diagrammatically shown on the display 5 of a portable device 1 (e.g. a smartphone).

Turning back to the data processing unit 3 of the device 1 and to the parameter generation mode of the physical activity rate parameter, it is worth noting that in an embodiment, such a data processing unit 3 is configured to execute the following operations.

The data process unit 3 is configured to calculate a target value of physical activity data to be generated (accumulated) by the user, at each set calculation time interval (e.g. every hour).

With respect to the actual amount of physical activity data generated with respect to the target value, the data processing unit 3 is configured to determine the plurality of intervals described above with particular reference to FIG. 3 (first interval RG1, second interval RG2, third interval RG3).

With this regard, the data processing unit 3 is configured to calculate the plurality of threshold values (e.g. the first threshold value S1 and the second threshold value S2) as follows.

Assuming a set number of hours of physical activity during a day, e.g. 12 hours, on the basis of experimental data, a set number of physical activity data, e.g. 1200, that a user with sporty profile can generate (accumulate) in the set number of hours of physical activity (12), is also available.

The data processing unit 3 is configured to divide the set number of physical activity data by the set number of hours of physical activity (1200/12=100) and subsequently divide such a result by the number of intervals of such a plurality of intervals of values of said physical activity rate parameter (three in the previously described example: first interval RG1, second interval RG2, third interval RG3). In this manner, the values of three intervals can be obtained: first interval RG1—from 0 to 33.3; second interval RG2—from 33.3 to 66.6; third interval RG3—from 66.6 to 99.9. In this case, the first threshold value S1 is 33.3, while the second threshold value S2 is 66.6. It is worth noting that the indicator 32 is positioned on the dashboard 31, for example, on an hourly basis.

In order to determine the number of hours of physical activity of the user, the data processing unit 3 is configured to set a time instant of beginning of physical activity by the user and an time instant of ending of physical activity by the user.

With this regard, in one embodiment, the time instant of beginning of physical activity is the time instant in which said at least one sensor 2 generates a set value of physical activity data (e.g. 5) in a set minimum period of time (e.g. in a period of time shorter than three minutes). The time instant of ending of physical activity is the time instant in which said at least one sensor 2 does not generate a set value of physical activity data (e.g. 5) in a set minimum period of time (e.g. three minutes).

According to a further embodiment, the time instant of beginning of physical activity and the time instant of ending of physical activity are obtained from the average of the time instants of beginning of physical activity and of the time instants of ending of physical activity, determined in an set sample time period (e.g. over the past two weeks).

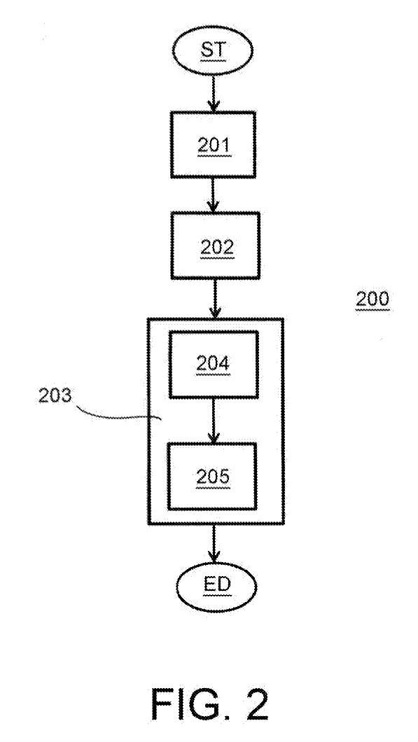

With reference now to FIG. 2, a method 200 for generating a parameter related to the physical activity of a user, in the following, also simply the method 200, is described.

The method 200 comprises a symbolic step of starting ST.

The method 200 further comprises a step of measuring 201, by said the sensor 2, information representative of the movement of a user.

Furthermore, the method 200 comprises a step of generating 202, by the sensor 2, on the basis of such information, a plurality of values of physical activity data.

The physical activity data are generated on the basis of such information, related to the intensity of physical activity performed by the user.

It should be noted that such an intensity of the physical activity may be expressed by the physiological measure expressing the energy cost of physical activities called MET (i.e. METabolic equivalent).

Turning back to FIG. 2, the method 200 further comprises a step of generating 203, by the data processing unit 3, at least one activity rate parameter on the basis of said plurality of values of physical activity data generated by the sensor 2.

Furthermore, the step of generating 203 comprises a step of generating 204, by the data processing unit 3, the activity rate parameter in a first reference time instant t0 on the basis of a portion of values of the plurality of values of physical activity data generated by said at least one sensor 2 in a set period of time preceding the first reference time instant t0.

The step of generating 203 further comprises a step of setting 205 such a set period of time preceding the first reference time instant t0 as any period of time preceding the first reference time instant t0, e.g. 1 hour before the first reference time instant t0, half hour before the first reference time instant t0, 10 minutes before the first reference time instant t0, 1 minute before the first reference time instant t0, and so on.

The method 200 ends with a symbolic step of ending ED.

According to a further embodiment, not shown in the figure, the step of setting 205 comprises a step of setting, by the data processing unit 3, the set period of time preceding the first reference time instant t0 as a period of time comprised between a time instant of beginning of physical activity (defined above) and the first reference time instant t0.

According to a further embodiment not shown in the figure, the method 200 further comprises a step of generating, by the data processing unit 3, a first graphic element 30 representative of the generated physical activity rate parameter.

In particular, the step of generating the first graphic element 30 comprises a step of comparing, by the data processing unit 3, the generated physical activity rate parameter with a set plurality of threshold values of the generated physical activity rate parameter. The set plurality of threshold values was defined above with particular reference to the embodiment of FIG. 3.

According to an embodiment, the method 200 subsequently comprises the step of placing, by the data processing unit 3, an indicator 32, relative to a dashboard 31 of the first graphic element 30, on the basis of the comparison of the generated physical activity rate parameter with the established plurality of threshold values of the generated physical activity rate parameter.

According to an embodiment, the method 200 further comprises a step of generating, by the data processing unit 3, a second graphic element 40 representative of the amount of values of physical activity data generated by said at least one sensor 2. The second graphic element 40 is described above.

In particular, the step of generating a second graphic element 40 comprises the steps of:
displaying, by the data processing unit 3, on a reference bar 41 a first progressive bar 42 representative of the amount of values of physical activity data generated by said at least one sensor 2 in a set current time instant tc (t0);
displaying, by the data processing unit 3, on the reference bar 41 a second progressive bar 43 representative of the amount of values of physical activity data generated by said at least one sensor 2 in the same current time instant tc (t0) of a preceding set period of time (e.g. the last two weeks).

According to a further embodiment, not shown in the figures, the method 200 comprises a step of generating, by the data processing unit 3, a third graphic element 50 representative of an informative sheet adapted to suggest to the user the physical exercise or how to improve the lifestyle. Such a sheet was described above.

According to a further embodiment, not shown in the figures, the method 200 comprises a step of generating, by the data processing unit 3, a fourth graphic element 60 representative of the amount of physical activity data generated by said at least one sensor 2 in the reference time instant t0. The fourth graphic element 60 was described above.

Furthermore, according to the present embodiment, not shown in the figures, the method 200 comprises the steps of generating, by the data processing unit 3:
a first portion of further graphic elements 61, close to the fourth graphic element 60, each of which representative of a physical exercise or physical activity which can be performed by the user, concurrently with the target set to the user related to the physical activity data to be generated and to a set period of time of performance of the physical exercise or physical activity;
a second portion of further graphic elements 62, each of which is representative of a physical exercise or physical activity that can be performed by the user, together with the progressive value of generated physical activity data and to the incremental value of the time of performance of the physical exercise or physical activity.

According to a further embodiment of the present invention, a program product can be loaded in the memory unit 4 of an electronic calculator (e.g. the portable device 1, provided with the data processing unit 2).

The program product can be executed by the data processing unit 3 of the electronic calculator (e.g. the data processing unit 3 of the portable equipment 1) to perform the steps of the method 200 for generating a parameter related to the physical activity of a user, previously described with reference to the embodiment in FIG. 2.

The portable device, the method and the program product according to the embodiments of the present disclosure allow the user to obtain an activity rate parameter representative of the physical activity performed by the user in a set period of time preceding the time instant in which such parameter is generated (the first reference time t0), such as, in the last hour, in the last half hour, in the last minutes, a period of time comprised between a beginning of physical activity time instant and the first reference time instant t0.

In other words, the user is provided at any time with an updated parameter related to the physical activity performed by the user herself/himself, who can obtain more precise, reliable information about the performed physical activity.

A person skilled in the art may produce several changes, adjustments and replacements of elements with other functionality equivalent ones to the embodiments of the device, method and program product for generating a parameter related to the physical activity of a user described above in order to meet incidental needs, without departing from the scope of the following claims. Each of the features described as belonging to a possible embodiment can be obtained independently of the other described embodiments.

1. A portable device for generating a parameter related to physical activity of a user, comprising:
at least one sensor for measuring information representative of movement of a user, said at least one sensor being further configured to generate, on the basis of the information, a plurality of values of physical activity data;
a data processing unit, operatively connected to said at least one sensor, configured to generate at least one physical activity rate parameter on the basis of said plurality of values of physical activity data generated by said at least one sensor;
the data processing unit being further configured to generate said at least one physical activity rate parameter in a first reference time instant on the basis of a portion of values of said plurality of values of physical activity data generated by said at least one sensor in a set period of time preceding the first reference time instant.

2. A device according to claim 1, wherein said set period of time preceding the first reference time instant may be stated as any period of time preceding the first reference time instant.

3. A device according to claim 1, wherein the set period of time preceding the first reference time instant is a period of time comprised between a time instant of beginning of physical activity and the first reference time instant.

4. A device according to claim 1, further comprising a memory unit, operatively associated to the data processing unit, said memory unit being configured to store both a program product to allow the device to generate the at least one physical activity rate parameter of the user and the data processed by the data processing unit during operation of the device, said memory unit being further configured to store the plurality of values of physical activity data generated by said at least one sensor and said at least one physical activity rate parameter generated by the data processing unit.

5. A device according to claim 1, further comprising a display configured to show to the user at least one of the following: the plurality of values of physical activity data generated by the at least one sensor and said at least one activity rate parameter generated by the data processing unit.

6. A device according to claim 1, further comprising a control panel configured to allow the user to interact with the portable device.

7. A device according to claim 1, further comprising a data communication module configured to communicate with other electronic devices and electronic calculators via a data communication channel in a wired or wireless mode.

8. A device according to claim 1, wherein the physical activity data are generated by said at least one sensor on the basis of the information related to intensity of the physical activity performed by the user.

9. A device according to claim 1, wherein said at least one sensor comprises an accelerometer, a gyroscope, a combination of a gyroscope and accelerometer, or any other electronic component able to measure information representative of movement of the user and to generate, on the basis of the information, a plurality of values of physical activity data.

10. A device according to claim 1, wherein the device is a smartphone, a tablet, a smartwatch, a personal digital assistant, a multimedia digital player or any other electronic equipment which can be worn by, or equip a user.

11. A device according to claim 5, wherein the data processing unit is configured to generate a first graphic element representative of the generated at least one physical activity rate parameter.

12. A device according to claim 10, wherein the data processing unit is configured to compare the generated at least one physical activity rate parameter with a set plurality of threshold values of the generated at least one physical activity rate parameter.

13. A device according to claim 5, wherein the data processing unit is further configured to generate a second graphic element representative of the amount of physical activity data values generated by said at least one sensor.

14. A device according to claim 13, wherein the data processing unit is configured to display on a reference bar a first progressive bar representative of the amount of values of physical activity data generated by said at least one sensor in a set current time instant, the data processing unit being configured to display on the reference bar a second progressive bar representative of the amount of values of physical data activity generated by said at least one sensor in a same current time instant of a preceding set period of time.

15. A device according to claim 5, wherein the data processing unit is further configured to generate a fourth graphic element representative of the amount of physical activity data generated by said at least one sensor during a set overall period of time.

16. A method for generating a parameter related to physical activity of a user, comprising the steps of:
measuring, by at least one sensor, information representative of movement of a user;
generating, by said at least one sensor, on the basis of such information, a plurality of values of physical activity data;
generating, by a data processing unit, at least one physical activity rate parameter on the basis of said plurality of physical activity data values generated by said at least one sensor; the step of generating comprises a step of generating, by the data processing unit, said at least one physical activity rate parameter in a first reference time instant on the basis of a portion of values of said plurality of values of physical activity data generated by said at least one sensor in a set period of time preceding the first reference time instant.

17. A method according to claim 16, wherein the step of generating comprises a step of generating, by the data processing unit, the at least one physical activity rate parameter in a first reference time instant on the basis of a portion of values of the plurality of values of physical activity data generated by said at least one sensor in a set period of time preceding the first reference time instant.

18. A method according to claim 16, wherein the step of generating further comprises a step of setting said set period of time preceding the first reference time instant as any period of time preceding the first reference time instant.

19. A method according to claim 18, wherein the step of setting comprises a step of setting, by the data processing unit, the set period of time preceding the first reference time instant as a period of time comprised between a time instant of beginning of physical activity and the first reference time instant.

20. A method according to claim 16, further comprising the step of generating, by the data processing unit, a first graphic element representative of the generated at least one physical activity rate parameter.

21. A method according to claim 20, wherein the step of generating the first graphic element comprises a step of comparing, by the data processing unit, the generated at least one physical activity rate parameter with a set plurality of threshold values of the generated at least one physical activity rate parameter.

22. A method according to claim 16, further comprising a step of generating, by the data processing unit, a second graphic element representative of the amount of physical activity data values generated by said at least one sensor.

23. A method according to claim 22, wherein the step of generating a second graphic element comprises the steps of:
displaying, by the data processing unit, on a reference bar, a first progressive bar representative of the amount of values physical activity data generated by said at least one sensor in a set current time instant;
displaying, by the data processing unit, on the reference bar, a second progressive bar representative of the amount of values of physical activity data generated by said at least one sensor in the same current time instant of a preceding set period of time.

24. A method according to claim 16, comprising a step of generating, by the data processing unit, a third graphic element representative of a sheet of a physical exercise which can be performed by a user equipped with the portable device.

25. A method according to claim 16, comprising a step of generating, by the data processing unit, a fourth graphic element representative of the amount of physical activity data generated by said at least one sensor during a set overall period of time.

26. A method according to claim 25, comprising the step of generating, by the processing unit:
a first portion of further graphic elements, close to the fourth graphic element, each of which is representative of a physical exercise or physical activity which can be performed by the user, together with a target set to the user related to the physical activity data to be generated and to a set period of time of performance of the physical exercise or physical activity;
a second portion of further graphic elements, each of which is representative of a physical exercise or physical activity to be performed by the user, together with a progressive value of generated physical activity data and with an incremental time value of performance of the physical exercise or physical activity.

27. A program product loadable in a memory unit of an electronic calculator, the program product being executed by a data processing unit of the electronic calculator to execute the steps of the method according to claim 16.
